(19) United States
(12) Patent Application Publication
(10) Pub. No.: US 2013/0122578 A1
(43) Pub. Date: May 16, 2013

(54) Fungus Diagnostic Detention Device

(71) Applicant: T. Todd Horak, New Lenox, IL (US)

(72) Inventor: T. Todd Horak, New Lenox, IL (US)

(21) Appl. No.: 13/296,851

(22) Filed: Nov. 15, 2011

(51) Int. Cl. C12M 1/34

(52) U.S. Cl. 435/288.7

(57) ABSTRACT

A fungus diagnostic device for detecting the presence of a fungus includes a testing container having an open top and defining an interior area configured to receive a protein formulated to promote fungus growth. The diagnostic device includes an invertible cap removably coupled to the container open top. A plunger is mounted to a top side of the cap.

The cap is movable between a pre-testing configuration with the plunger outside of the testing container and a testing configuration in which the plunger is inside the testing container interior area. A gelatinous protein is situated in the interior area of the testing container and formulated to change color in the presence of a fungus. The diagnostic device includes an electrical plug electrically connected to an electrical power source. A heating element is situated adjacent the container and electrically connected to the heating element.

### BACKGROUND OF THE INVENTION

This invention relates generally to devices that diagnose medical conditions and, more particularly to a handheld device for diagnosing if a toenail includes a fungus.

The growth of fungus under or in a person's toenail is an unsightly and potentially dangerous condition. If a person suspects that a fungus is growing in or under one of his toes, he typically goes to his doctor or to the hospital for diagnosis and treatment. The process of diagnosing this condition beyond speculation during a doctor's examination usually involves removing a fragment of the toenail and then subjecting it to a chemical test for a period of about 10 to 14 days. The diagnostic test usually includes applying a protein substance to the nail fragment and then observing if a fungus grows perceptibly on the toenail. Specifically, the diagnostic test seeks to create an environment conducive to fungus growth. If there is no fungus on or in the toenail, of course, no fungus will grow even in the artificially fungus-supporting environment.

Although assumably effective for their intended purposes, the existing diagnostic testing procedures and equipment in the prior art typically require 10-14 days of testing and monitoring within a lab or medical facility. The delay in receiving a result causes the actual fungus—if it exists—to flourish even more before treatment begins.

Therefore, it would be desirable to have a fungus diagnostic device that is handheld and portable and configured to conveniently test a toenail fragment or fingernail fragment in a home environment. Further, it would be desirable to have a fungus diagnostic device having a testing container that includes an appropriate testing substance and includes a plunger for inserting a toenail or fingernail into the testing container for diagnostic testing. In addition, it would be desirable to have a fungus diagnostic device configured to impart heat to the testing container so that the speed of diagnosing the fungus is increased.

### SUMMARY OF THE INVENTION

A fungus diagnostic device for use in testing for the presence of a fungus in a person's toenail includes a testing container having an open top and defining an interior area configured to receive a protein formulated to promote fungus growth. The diagnostic device includes an invertible cap removably coupled to the container open top. A plunger is mounted to a top side of the cap. A plunger is coupled to a top of the cap. The cap is movable between a pre-testing configuration in which the plunger is outside of the testing container and a testing configuration in which the plunger is inside the testing container interior area. A gelatinous protein substance is situated in the interior area of the testing container, the protein substance formulated to change color in the presence of a predetermined fungus. The diagnostic device includes an electrical plug electrically connected to an electrical power source. A heating element is situated adjacent the container and electrically connected to the heating element.

Therefore, a general object of this invention is to provide a fungus diagnostic device for testing a toe nail (or fingernail) to determine if it has fungus growing on it so that appropriate medical or sanitizing action may be taken.

Another object of this invention is to provide a fungus diagnostic device, as aforesaid, having a testing container that includes a protein gel into which a toenail fragment may be inserted for diagnostic testing.

Still another object of this invention is to provide a fungus diagnostic device, as aforesaid, having one or more heating elements to provide heat to the testing container to speed up the diagnostic process.

Yet another object of this invention is to provide a fungus diagnostic device, as aforesaid, having a timer to indicate when a diagnostic process is complete.

A still further object of this invention is to provide a fungus diagnostic device, as aforesaid, having a timer display and a speaker for providing audio and visual indications of when a diagnostic process is complete.

A still further object of this invention is to provide a fungus diagnostic device, as aforesaid, that is user-friendly to use.

Other objects and advantages of the present invention will become apparent from the following description taken in connection with the accompanying drawings, wherein is set forth by way of illustration and example, embodiments of this invention.

### BRIEF DESCRIPTION OF THE DRAWINGS

FIG. 1 is a perspective view of a fungus diagnostic device according to a preferred embodiment of the present invention;

FIG. 2 is a perspective view of the fungus diagnostic device removed from the housing as in FIG. 1 illustrated with the plunger and cap in an outside the testing container configuration;

FIG. 3 is a perspective view of the fungus diagnostic device as in FIG. 2 illustrated with the plunger and cap in an inside the testing container configuration;

FIG. 4A is a front view of the fungus diagnostic device as in FIG. 1; and

FIG. 4B is a sectional view taken along line 4B-4B of FIG. 4A.

### DESCRIPTION OF THE PREFERRED EMBODIMENT

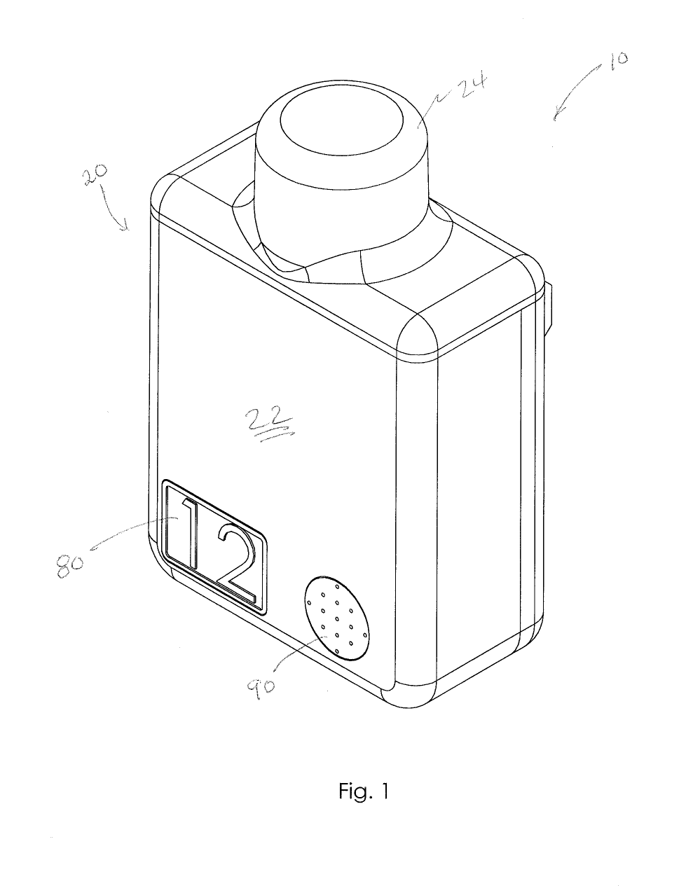

A toenail fungus diagnostic device 10 according to a preferred embodiment of the present invention will now be described with reference to FIGS. 1 to 4b of the accompanying drawings. The diagnostic device 10 includes a testing container 30, heating element 50, and an electrical plug 60 for electrically communicating the heating element 50 to an electrical power source.

The diagnostic device 10 may include a housing 20 having a bottom, upstanding side walls, and a top configured to receive all of the components described below (FIG. 1). All of the components described below are preferably situated in the housing 20 unless specifically noted otherwise. The front wall 22 of the housing 20 may define openings or windows through which other components may be seen. In addition, at least the front wall 22 of the housing 20 may be constructed of a transparent material. The housing 20 may include a lid 24 or cover that may be removed to provide access to the housing interior area, i.e. to access the components to be described below.

The diagnostic device 10 may include a testing container 30 having a continuous side wall 32 extending upwardly from a bottom wall 34. The bottom 34 and side 32 walls are integrally connected and define an open interior area. An upper edge of the side wall 32 defines an open top. An invertible cap 36 having bottom 38 and top 39 sides is configured to attach to the testing container 30 and to selectively provide access to the interior area when removed therefrom or to prevent access to the open interior area when coupled thereto. The cap 36 may be coupled to the testing container 30 with threads, a friction fit construction, or with other fasteners (not shown).

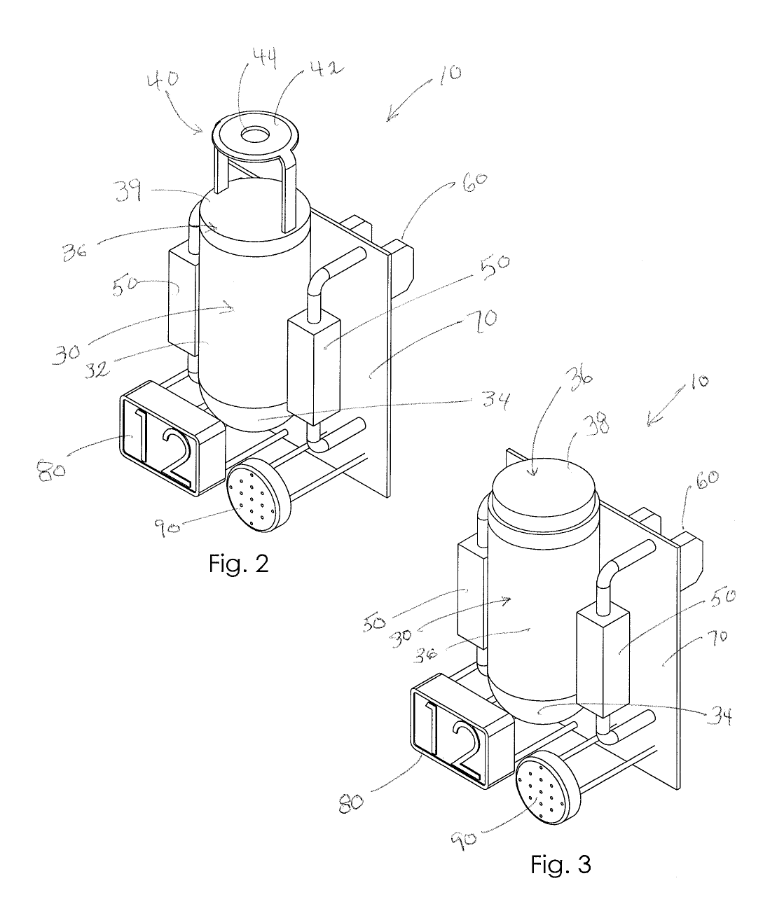

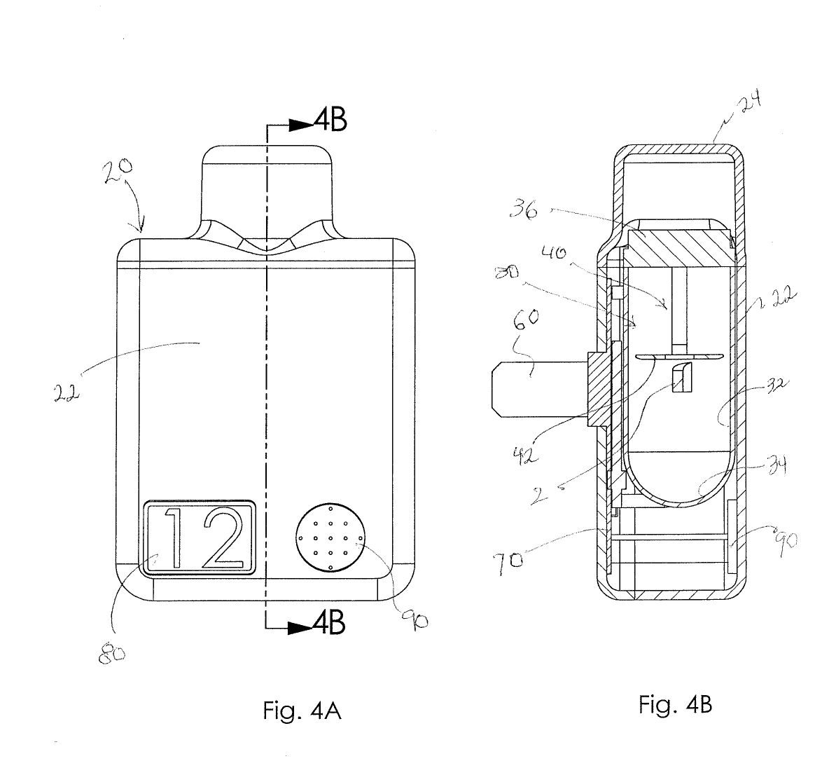

A plunger 40 is fixedly attached to the top side 38 of the cap 36 and extends away therefrom (FIG. 2). The cap 36 is invertible between a pre-testing configuration in which the plunger 40 is outside of the testing container 30 (FIG. 2) and a testing configuration in which the plunger 40 is situated inside the interior area of the testing container 30 (FIG. 3). As will be described later in relation to using the device 10, the plunger 40 is useful to push a toenail fragment 2 deep into the fungus testing substance within the interior area of the testing container 30 (FIG. 4b).

The fungus testing substance situated inside the container 30 may be a gelatinous protein being formulated to promote or foster fungus growth. This formulation has utility in that if a predetermined type or types of fungus/fungi are deposited into the protein environment, the fungus will continue to grow and, actually, to grow more rapidly than in an open air or dry environment. The fungus testing substance may be formulated to change color when in contact with predetermined types of fungus. Preferably, then, the testing container 30 is constructed of transparent materials such that a user may visually recognize that a fungus has been detected on the toenail fragment and, as a result, take appropriate remedial actions to treat the fungus.

The plunger 40 may include a configuration specifically designed to produce a fluid current or mixing action of the testing substance when the plunger 40 is first inserted into the testing container 30. More particularly, the plunger 40 may include a platform 42 having a generally planar configuration that defines an aperture 44. It has been found that the planar configuration of the platform 42 causes a downward pressure upon both a toenail fragment so as to force the testing substance downwardly toward the bottom wall 34 which results in a backflow of the testing substance upwardly along the side wall 32. The aperture 44 enables a portion of the testing substance to pass upwardly therethrough so as to relieve the pressure created by insertion of the plunger into the interior area of the container 30.

The fungus diagnostic device 10 may include a circuit board 70 situated adjacent the testing container 30. The circuit board 70 may include circuitry configured to operate various electronic components of the present invention as will be described in due course below. It is understood that the circuit board 70 shown in the figures may be a structural member and the circuitry may be in the form of wires and other electrical components.

The fungus diagnostic device 10 includes an electrical plug 60, such as a plug-in having two or three metal prongs. The electrical plug 60 may be electrically connected to the circuit board 70 and extend rearwardly therefrom (FIGS. 2 and 3). The electrical plug 60 is configured to selectively interface with an electrical power source such as a wall outlet (not shown). Electricity is communicated from the electrical power source to the circuit board 70 when the electrical plug 60 is inserted into the power source (i.e. wall outlet).

The diagnostic device 10 may include at least one heating element 50 positioned adjacent the testing container 30 so as to provide heat to the testing container 30 when energized. As shown in the drawings, the heating element 50 may be situated on the circuit board 70 from which current for operation may be drawn. Preferably, the heating element 50 is electrically energized automatically when the electrical plug 60 is electrically connected to the power source as discussed above. The diagnostic device 10 may include another heating element, which is indicated using the same reference numeral as the heating element first described. The additional heating element 50 may be positioned on an opposite side of the testing container 30 for more complete and efficient heating thereof.

The fungus diagnostic device 10 may also include a timer (not shown as it is circuitry on the circuit board) electrically connected to the electrical plug 60. The timer may be a component on the circuit board 70 or including specific wiring as would be known in the art. The timer may be configured to be automatically actuated to count or measure a predetermined amount of time when it is electrically energized by electricity from the power source. In some embodiments, however, the timer may have an actuation button configured to manually initiate the timer operation. In use, the timer is used to estimate an amount of time for a toenail fragment to be tested for the existence of fungus as described previously.

The fungus diagnostic device 10 may also include a display device 80 electrically connected to the electrical plug 60 via the circuit board 70 and, more particularly, to the timer circuit. The display device 80 is in electrical communication with the timer and may be configured to display a current time measurement of the timer. The display device 80 may extend forwardly of the circuit board 70 and extend through a front wall of the housing 20.

The fungus diagnostic device 10 may also include a speaker 90 electrically connected to the circuit board 70 and, specifically, to the timer. The speaker 90 is configured to emit an audible sound, such as a beep, series of beeps, or other alarm sound, when the timer has reached a predetermined amount of time since its actuation. A audible sound by the speaker 90 may indicate that a toenail fragment has had a sufficient amount of time in the testing substance in order to determine if a fungus is present. Operation of the speaker 90 will enable a user to determine visually if the testing substance has changed colors indicative of the presence of a fungus on the toenail. The speaker 90 may extend forwardly of the circuit board 70 and extend through a front wall 22 of the housing 20.

In use, a user may gain access to the interior of the housing 20 by removing the lid 24 therefrom. The cap 36 of the testing container 30 may originally be configured with the plunger 40 extending upward from the top side 39 thereof. Accordingly, the user may remove the cap 36 in order to gain access to the testing container 30. With the cap 36 removed, a user may deposit a toenail fragment 2 (or fingernail fragment for that matter) into the testing container 30 where it will likely float atop the testing substance which may have a thick gel consistency. The cap 36 may then be inverted and inserted plunger first into the testing container 30. As the cap 36 is urged downwardly and simultaneously urges the toe nail downwardly, the testing substance moves in a mixing motion to fully saturate and cover the toenail. The cap 36 is then secured in the downwardly oriented configuration (FIGS. 3 and 4B).

At the inserted configuration, the device 10 may be plugged in to an electrical outlet, causing the heating element(s) 50, timer, display device 80, and speaker 90 to be energized. As the timer counts up (or down depending on the circuitry), a current time is displayed on the display device 80. The speaker 90 is actuated when the timer reaches a predetermined elapsed time, indicating that the fungus test is complete. A user may then visually inspect the color of the testing substance to see if it has changed color indicative of the existence of a fungus. If, in fact, a fungus has been detected, a user may take appropriate steps to rid his toenail of the fungus.

Accordingly, the fungus diagnostic device 10 provides means for individuals to test their own toenails or fingernails for the existence of a fungus without going to a doctor's office or other medical facility. In addition, the fungus diagnostic device 10 enables a user to speed up the detection process by simply plugging the hand-held device into a common electrical outlet.

It is understood that while certain forms of this invention have been illustrated and described, it is not limited thereto except insofar as such limitations are included in the following claims and allowable functional equivalents thereof.

1. A fungus diagnostic device for use in testing for the presence of a fungus on a person's toenail, comprising:
a testing container having a bottom wall and a continuous side wall extending upwardly therefrom, an upper edge of said side wall defining an open top, said bottom wall and side wall together defining an open area;
an invertible cap having top and bottom sides and removably coupled to said open top;
a plunger coupled to said top side of said cap and extending away;
wherein said cap is configured to be inverted between a pre-testing configuration in which said plunger is outside of said testing container and a testing configuration in which said plunger is inside said testing container interior area;
a fungus testing substance situated in said interior area of said testing container, said fungus testing substance formulated to change color in the presence of a predetermined fungus;
an electrical plug configured to selectively electrically connect with an electrical power source; and
a heating element situated adjacent said container and electrically connected to said electrical plug and configured to be energized automatically when said electrical plug is in electrical communication with said electrical power source.

2. The diagnostic device as in claim 1, further comprising a timer electrically connected to said electrical plug that is actuated to measure a predetermined amount of time when energized.

3. The diagnostic device as in claim 2, further comprising a display device electrically connected to said timer and configured to display a current measurement of time according to said timer.

4. The diagnostic device as in claim 3, further comprising circuitry that includes said timer and that is electrically connected to said display, said circuitry being electrically connected to said electrical plug so as to be energized when said electrical plug is electrically engaged with said electrical power source.

5. The diagnostic device as in claim 4, further comprising a speaker electrically connected to said speaker and configured to emit a sound when said timer reaches said predetermined amount of time.

6. The diagnostic device as in claim 5, further comprising a housing configured to contain said testing container, said circuitry, said timer, said heating element, and said speaker, said housing and said testing container having a transparent construction.

7. The diagnostic device as in claim 5, further comprising another heating element situated adjacent said testing container, said testing container being sandwiched between said heating element and said another heating element such that heat is imparted to two sides of said testing container when said heating element and said another heating element are energized.

8. The diagnostic device as in claim 6, wherein said electrical plug extending outwardly though a back wall of said housing.

9. The diagnostic device as in claim 1, further comprising another heating element situated adjacent said testing container, said testing container being sandwiched between said heating element and said another heating element such that heat is imparted to two sides of said testing container when said heating element and said another heating element are energized.

10. The diagnostic device as in claim 5, wherein said fungus testing substance is a gelatinous protein.

11. The diagnostic device as in claim 1, wherein said fungus testing substance is a gelatinous protein.

12. The diagnostic device as in claim 5, wherein said plunger includes a platform displaced from said cap, said platform having a generally planar configuration and defining an aperture so as to cause a mixing action of said testing substance when said plunger is moved toward said testing configuration inside said testing container.

13. The diagnostic device as in claim 1, wherein said plunger includes a platform displaced from said cap, said platform having a generally planar configuration and defining an aperture so as to cause a mixing action of said testing substance when said plunger is moved toward said testing configuration inside said testing container.
